(19) United States
(12) Patent Application Publication
(10) Pub. No.: US 2002/0065475 A1
(43) Pub. Date: May 30, 2002

(54) Medical guide wire and balloon catheter

(75) Inventors: Taiichiroh Meguro, Sendai-shi (JP); Masashi Momota, Nagoya-shi (JP); Tomihisa Kato, Nagoya-shi (JP)

Correspondence Address: MORGAN LEWIS & BOCKIUS LLP, 1111 PENNSYLVANIA AVENUE NW, WASHINGTON, DC, 20004, US

(73) Assignee: C-I-MEDIC Co., Ltd. and Asahi Intecc Co., Ltd.

(21) Appl. No.: 09/995,789

(22) Filed: Nov. 29, 2001

(30) Foreign Application Priority Data
Jan. 31, 2000 (JP) 2000-21298

(51) Int. Cl. A61M 25/00

(52) U.S. Cl. 600/585; 606/194

(57) ABSTRACT

In a medical guide wire 1, a rear half of a leading bulge portion (ellipsoidal helical spring) 5 forms a truncated cone shaped front catheter engagement portion 8. An inner wall of a balloon catheter 2 forms a spiral groove 9. The spiral groove 9 fits into the front catheter engagement portion 8 to provisionally connect the balloon catheter 2 to the front catheter engagement portion 8. The medical guide wire 1 and the balloon catheter 2 are inserted into the blood vessel with one single step procedure when the medical guide wire 1 is introduced into the blood vessel to place the balloon portion 10 at the stricture blood vessel area (P) to cure the stricture blood vessel area (P).

### BACKGROUND OF THE INVENTION

### FIELD OF THE INVENTION

The invention relates to a medical guide wire used at the time when a balloon catheter is placed at a stricture blood vessel area (coronary artery) to cure the stricture blood vessel area, and particularly concerns to a balloon catheter used in combination with the medical guide wire.

### DESCRIPTION OF PRIOR ART

Upon treating a stricture blood vessel area, a very thin flexible medical guide wire is inserted into the stricture blood vessel area to place a leading guide wire end past the stricture blood vessel area.

Thereafter, a tubular balloon catheter are introduced along the guide wire to reach the stricture blood vessel area when manipulating the guide wire grip exposed outside the blood vessel.

Then, a balloon portion of balloon catheter is inflated due to a liquid (e.g., physiological saline solution) supplied to the balloon catheter to forcibly expand the stricture blood vessel area. This permits a normal amount of blood to run through the blood vessel so as to remedy the stricture blood vessel area. By way of illustration, this is exemplified by a Japanese Provisional Utility Model Publication No. 5-19078.

In the prior remedial method in which the medical guide wire is inserted into the blood vessel, and thereafter the balloon catheter is introduced along the guide wire into the stricture blood vessel area, two step procedures are required to inflate the balloon portion. These are inserting the guide wire and introducing the balloon catheter into the stricture blood vessel area along the guide wire. This is time-consuming with the two step procedures and would aggravate pains which the patient feels intrusive during the manipulating the medical guide wire within the blood vessel.

Further, in the case which caps the medical guide wire with the balloon catheter that is diametrically greater than the medical guide wire, an open edge the balloon catheter forms a stepped section against the medical guide wire.

The stepped section would become an obstacle upon inserting the guide wire into the stricture blood vessel area. This is more time-consuming because the manipulator must take care so that the stepped section hitches the blood vessel and stricture blood vessel area. Otherwise, the stepped section would injure the blood vessel and/or the stricture blood vessel area by hitching them.

Therefore, the present invention has been made with the above drawbacks in mind, it is a main object of the invention to provide a medical guide wire combined with a balloon catheter which is capable of inserting the medical guide wire into a blood vessel and a stricture blood vessel area smoothly and quickly without injuring them, thus achieving remedial procedures quickly with ease and safety.

### SUMMARY OF THE INVENTION

According to the present invention, there is provided a medical guide wire having an elongated and flexible core member, a front catheter engagement portion provided around the core member and formed into a bulged shape or a truncated cone shape, a diameter of which progressively decreases as approaching a rear end of the front catheter engagement portion. The front catheter engagement portion is capped with the balloon catheter to provisionally connect the balloon catheter to the front catheter engagement portion so that the balloon catheter is introduced into a blood vessel together with the medical guide wire.

According to other aspect of the invention, a provisionally connecting member is provided at a front open end of the balloon catheter to provisionally connect the balloon catheter to the front catheter engagement portion.

The provisionally connecting member is a carve in the form of kerf, slot, notch or slit defined at the front open end of the balloon catheter. Otherwise, the provisionally connecting member is a rolled end or a spiral groove which fit into the leading bulge portion formed by the ellipsoidal helical spring.

According to other aspect of the invention, the balloon catheter and the front catheter engagement portion are formed by a common synthetic resin to produce a coefficient of static friction therebetween, a magnitude of which is determined enough to provisionally connect the balloon catheter to the front catheter engagement portion.

With the medical guide wire capped with the balloon catheter assembled before introducing into the blood vessel, the assemble of the medical guide wire and the balloon catheter act as a leading head portion to guide the guide wire into the blood vessel. By concurrently inserting the medical guide wire and the balloon catheter into the blood vessel, it is possible to place a balloon portion of the balloon catheter at the stricture blood vessel area with one single step procedure.

After inflating the balloon portion at the stricture blood vessel area, it is necessary to withdraw the balloon catheter from the medical guide wire to replace the balloon portion in turn with larger ones. For this purpose, the provisionally connecting member is provided to separate the balloon catheter from the medical guide wire by simply pulling a rear end of the balloon catheter exposed outside the blood vessel.

The front catheter engagement portion is in integral with the leading bulge portion to readily introduce the leading bulge portion into the stricture blood vessel area.

As other alternatives, the front catheter engagement portion is connected in series with the leading bulge portion, and connected to the leading bulge portion by means of a soldering or an adhesive.

As the provisionally connecting member, the front catheter engagement portion and the balloon catheter are formed by the common synthetic resin to determine the coefficient of the static friction therebetween, a magnitude of which is great enough to provisionally connect the balloon catheter to the front catheter engagement portion.

With the medical guide wire thus combined with the balloon catheter, it is possible to concurrently introduce the balloon catheter and the medical guide wire into the stricture blood vessel area at the time when leading the medical guide wire into the stricture blood vessel area.

This enables a manipulator to attain the balloon catheter to the stricture blood vessel area with one single step procedure so as to quickly prepare for the treatment more than the prior art which requires the two step procedures to lead the balloon catheter into the stricture blood vessel area.

With the use of the provisionally connecting member, it is possible to smoothly advance the guide wire into the complicatedly turned blood vessel without slipping the balloon catheter off the guide wire, and further replacing the balloon catheter easily with larger ones.

With the front catheter engagement portion leading the balloon catheter into the stricture blood vessel area, it is possible to prevent the blood vessel and the stricture blood vessel area from getting injured due to the front open edge of the balloon catheter. This also facilitates to place the balloon catheter at the stricture blood vessel area with safety and ease.

### BRIEF DESCRIPTION OF THE DRAWINGS

A preferred form of the present invention is illustrated in the accompanying drawings in which:

FIG. 1 is a longitudinal cross sectional view of a medical guide wire and a balloon catheter according to a first embodiment of the invention;

FIG. 2 is a longitudinal cross sectional view of the medical guide wire combined with the balloon catheter;

FIG. 3 is a longitudinal cross sectional view shown to explain how to manipulate the medical guide wire;

FIG. 4 is a longitudinal cross sectional view of the medical guide wire combined with the balloon catheter according to a second embodiment of the invention;

FIG. 5 is a longitudinal cross sectional view of the medical guide wire capped with the balloon catheter according to a third embodiment of the invention;

FIG. 6 is a longitudinal cross sectional view of the medical guide wire combined with the balloon catheter according to a fourth embodiment of the invention;

FIG. 7 is a longitudinal cross sectional view of the medical guide wire combined with the balloon catheter according to a fifth embodiment of the invention;

FIG. 8 is a latitudinal cross sectional view taken along the line VIII-VIII of FIG. 7;

FIG. 9 is a longitudinal cross sectional view of the medical guide wire combined with the balloon catheter according to a sixth embodiment of the invention;

FIG. 10 shows a way how the medical guide wire is introduced into the blood vessel together with the balloon catheter; and

FIG. 11 shows another way how the medical guide wire is introduced into the blood vessel separately from the balloon catheter.

### DETAILED DESCRIPTION OF THE PREFERRED EMBODIMENTS

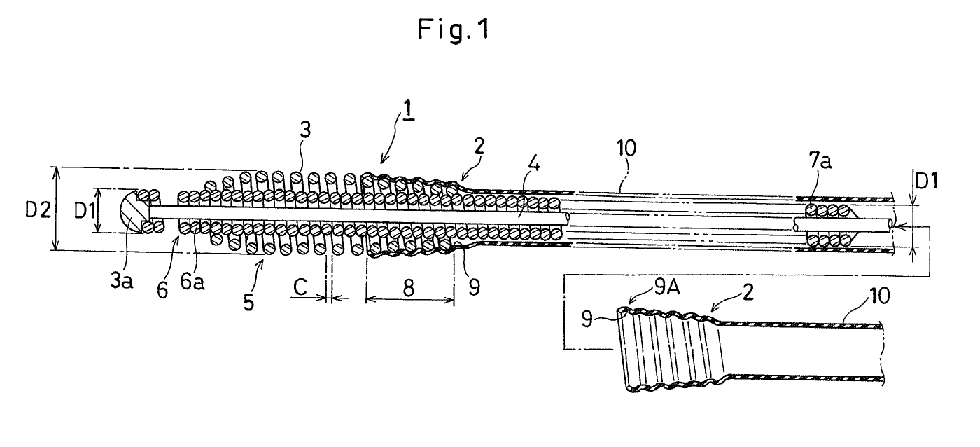

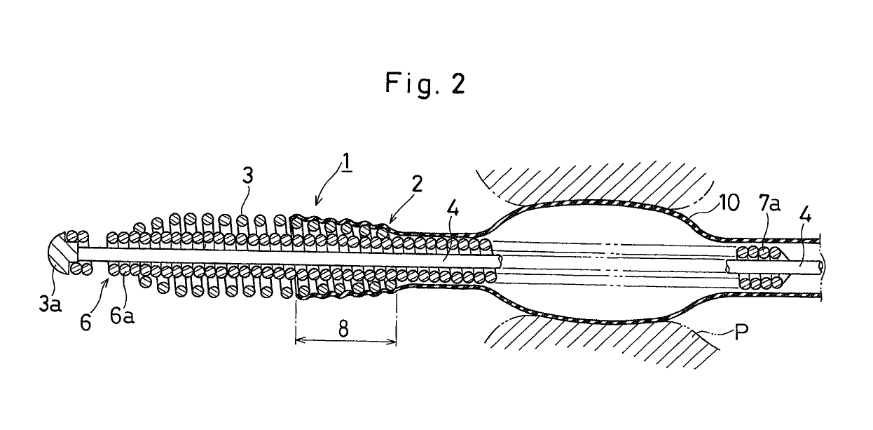

Referring to FIGS. 1 and 2 which show a medical guide wire 1 and a tubular balloon catheter 2 used to the medical guide wire 1 according to a first embodiment of the invention, a helical spring 6 is coiled around an elongated and flexible core member 4, a front end of which has a head portion 3a.

The helical spring 6 has a diameter (D1) which is slightly greater than that of the core member 4, and has line elements tightly arranged along the core member 4 with no clearance appeared between the line elements.

Around a front portion of the helical spring 6, an ellipsoidal helical spring 3 is provided to form a leading bulge portion 5. The ellipsoidal helical spring 3 has a maximum diameter (D2), a rear half of which forms a front catheter engagement portion 8 shaped into a truncated cone configuration, a diameter of which progressively decreases as approaching a rear end of the front catheter engagement portion 8.

The leading bulge portion 5 acts as a soft and flexible guide member effective when the medical guide wire 1 is inserted into a stricture blood vessel area (P).

In the leading bulge portion 5, the ellipsoidal helical spring 3 is fixed to the helical spring 6 by means of a soldering or an adhesive. The helical spring 6 has a forward portion 6a and a rearward portion 7a each connected consecutively along the core member 4. The ellipsoidal helical spring 3 has line elements arranged over the helical spring 6 with a certain clearance (C) appeared between the line elements. The ellipsoidal helical spring 3 forms the front engagement portion 8 extending from the maximum diameter section (D2) to a boundary area in which the maximum diameter section (D2) start to descend to the small diameter section (D1).

The balloon catheter 2 is formed by a flexible synthetic resin, and an inner wall of the balloon catheter 2 has a spiral groove 9 to serve as a connecting member 9A. By pushing an open front end of the balloon catheter 2 against the front engagement portion 8, the spiral groove 9 fits into the line elements of the ellipsoidal helical spring 3 to provisionally (detachably) connect the balloon catheter 2 to the front engagement portion 6.

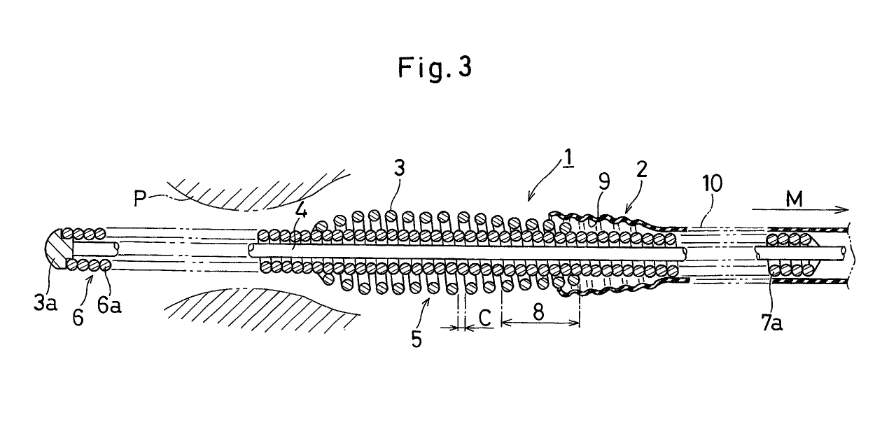

Upon inserting the medical guide wire 1 into a blood vessel, the leading bulge portion 5 advances into the blood vessel together with the balloon catheter 2 to expand the stricture blood vessel area (P) so as to place a balloon portion 10 at the stricture blood vessel area Then, a physiological liquid is injected into the balloon portion 10 to inflate it so as to expand the stricture blood vessel area (P) as shown in FIG. 2. When the balloon portion 10 is replaced with a larger one, the balloon catheter 2 is pulled in the direction of an arrow M to such a degree as to move the leading bulge portion 5 behind the stricture blood vessel area (P) as shown in FIG. 3. By fully pulling a rear grip portion (not shown) exposed outside the blood vessel, the balloon catheter 2 separates the leading bulge portion 5 to be withdrawn from the leading bulge portion 5.

When the balloon portion 10 is replaced by a still larger one, a diameter-increased balloon catheter is introduced along the medical guide wire 1 into the blood vessel to provisionally connect the balloon catheter to the front catheter engagement portion 8 so as to lead the new balloon portion into the stricture blood vessel area (P). Thus, the balloon portions are replaced in turn by larger ones.

It is noted that the spiral groove 9 is formed by forcibly fitting the balloon catheter 2 into the front catheter engagement portion 8 to heat the balloon catheter 2 before cooling it down.

In this instance, the front catheter engagement portion 8 approximately measures 0.56 mm in maximum diameter, 0.355 mm in minimum diameter and 2.0 mm in length. The forward portion 6a of the helical spring 6 approximately measures 40.0 mm in length, the leading bulge portion 5 approximately measures 5.0 mm in length while the helical spring 6 approximately measures 300 mm in length.

With the structure thus described, it is possible to insert the medical guide wire 1 into the blood vessel in combination with the balloon catheter 2. In addition, the front catheter engagement portion 8 is formed in integral with the leading bulge portion 5. This eliminates the necessity of providing a discrete front catheter engagement portion, thus conducing to simplifying a whole structure with a lower cost.

With the clearance (C) appeared between the line elements of the ellipsoidal helical spring 3, the clearance (C) admits the blood passage running through the stricture blood vessel area (P) when the leading bulge portion 5 is placed at the stricture blood vessel area (P).

Under the presence of the clearance (C), it is possible to determine a greater lead pitch between the line elements of the ellipsoidal helical spring 3. The greater lead pitch enables a manipulator to a longer travel when the leading bulge portion 5 is rotated to move it past the stricture blood vessel area (P) with the ellipsoidal helical spring 3 engaged against an inner wall of the stricture blood vessel area (P). This quickly advances the leading bulge portion 5 to easily place the balloon portion 10 at the stricture blood vessel area (P) By moving the leading bulge portion 5 back and forth through the stricture blood vessel area (P), remnants deposited on the inner wall of the stricture blood vessel area (P) are partly removed to widely open the stricture blood vessel area (P). The widely opened stricture blood vessel area (P) makes it easier to introduce the balloon portion 10 into the stricture blood vessel area (P), thus conducing to smoothly placing the balloon portion 10 at the stricture blood vessel area (P) with ease and safety.

When a stent already retained in the stricture blood vessel area (P) is abnormally deformed, the medical guide wire 1 can be introduced together with the balloon catheter 2 into the blood vessel to treat the stricture blood vessel area (P) again. The leading bulge portion 5 moves into the stent to gradually rectify the deformed stent so as to facilitate the treatment appropriately as opposed to the prior art in which the balloon catheter hitches the deformed stent to block its passage through the stricture blood vessel area (P).

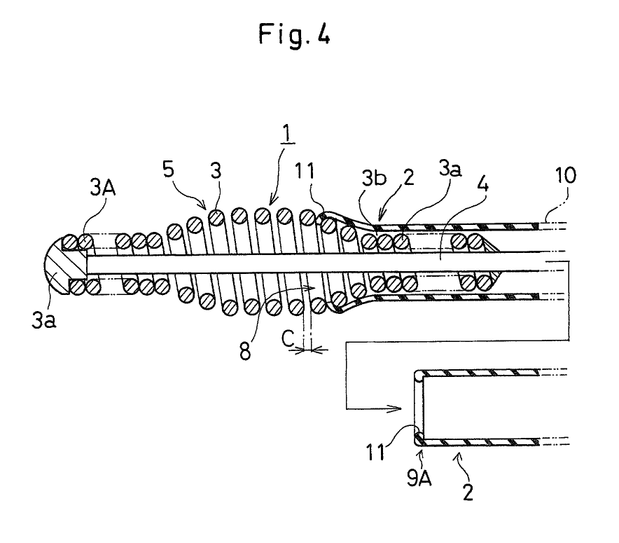

FIG. 4 shows a second embodiment of the invention in which the provisionally connecting member 9A is formed on an open front end of the balloon catheter 2.

The open front end of the balloon catheter 2 is rolled inward to define a rolled end 11. The rolled end 11 fits into the clearance (C) of the ellipsoidal helical spring 3 which is sandwiched between a diameter-reduced forward helical spring 3A and a diameter-reduced rearward helical spring 3a to provisionally connect the balloon catheter 2 to the front catheter engagement portion 8.

Instead of fitting into the clearance (C), the rolled end 11 can fit into an annular cavity 3b formed between the tightly arranged line elements of a diameter-reduced reward helical spring 3a to provisionally connect the balloon catheter 2 to the front catheter engagement portion 8.

Due to the absence of an outwardly directed edge usually defined on the open front end of the balloon catheter 2 and detrimental to the blood vessel when inserting the balloon catheter 2 into the blood vessel, the medical guide wire 1 can be safely inserted into the blood vessel together with the balloon catheter 2.

Considering that the rolled end 11 is formed by pushing the open front end of the heated balloon catheter 2 against a die mold before cooling down the balloon catheter 2, a molecular orientation caused by rolling a crystallized resin hypotube is tempered to alleviate the mechanical anisotropy. This allows the rolled end 11 to restore the appropriate flexibility so as to render the provisionally connecting member 9A functionally effective.

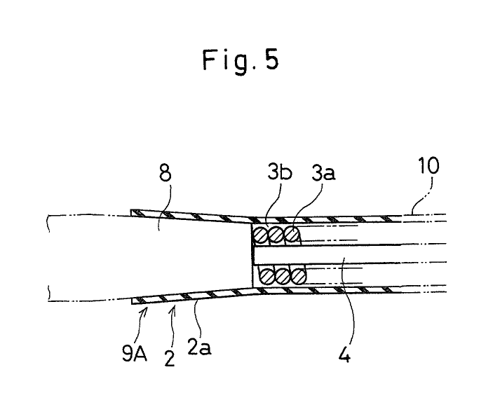

FIG. 5 shows a third embodiment of the invention in which the front catheter engagement portion 8 is in the form of a truncated cone configuration, a diameter of which progressively decreases as approaching the helical spring 3a coiled around the core member 4.

The front catheter engagement portion 8 and the balloon catheter 2 are formed commonly by a polyamide-based synthetic resin. For example, the balloon catheter 2 is formed by polyamide, and the front catheter engagement portion 8 by polyimide or hot melt adhesive (a.k.a. Bestamelt Adhesive as a trade name). A front section 2a of the balloon catheter 2 is forcibly fit over the front catheter engagement portion 8 to provisionally connect the balloon catheter 2 to the front catheter engagement portion 8 to serve as the provisionally connecting member 9A.

For this reason, a coefficient of static friction between the front section 2a of the balloon catheter 2 and the front catheter engagement portion 8 is determined to be such a magnitude as to provisionally connect the balloon catheter 2 to the front catheter engagement portion 8.

In this instance, at least, the front section 2a of the balloon catheter 2 and the front catheter engagement portion 8 are preferably formed by polyamide, polyvinyl chloride, polytetrafluoroethylene or polyethylene.

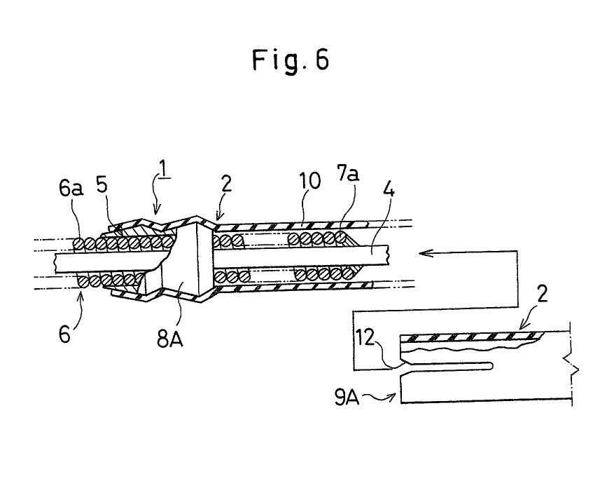

FIG. 6 shows a fourth embodiment of the invention in which a front catheter engagement portion 8A is formed around the helical spring 6 into a gourd-shaped (columnar-shaped) configuration. The front catheter engagement portion 8A is mirror finished after molding it from the hot melt adhesive based on polyamide, polyethylene or the like (thermally sensitive adhesive) or silicone-based adhesive (reactive type adhesive).

An open end section of the balloon catheter 2 has a pair of diametrically opposed axial carves 12 in the form of a kerf, slit, slot or notch. The axial carves 12 help expand the balloon catheter 2 when fitting the balloon catheter 2 over the front catheter engagement portion 8A to provisionally connect them readily.

The adhesive materials makes it possible to soften so as to the front catheter engagement portion 8A more flexible when the medical guide wire 1 is manipulated. The mire finish treatment renders the balloon catheter 2 to tightly attach to the front catheter engagement portion 8A so as stabilize the function of the provisionally connecting member 9A.

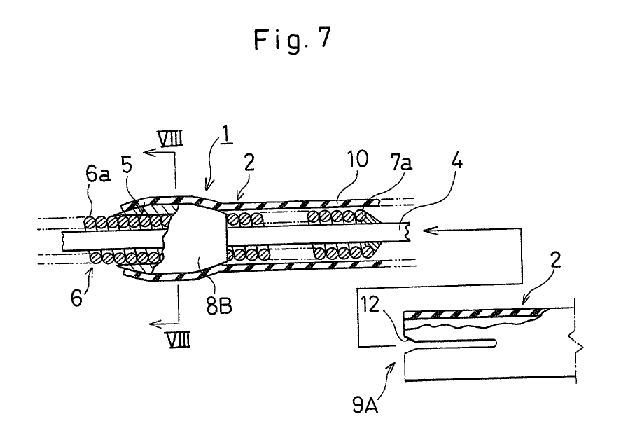

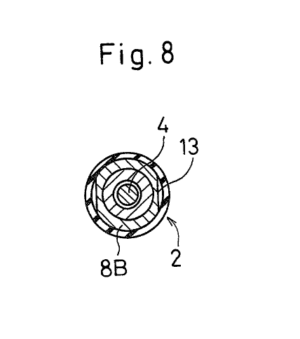

FIGS. 7 and 8 show a fifth embodiment of the invention which differs from the fourth embodiment in that a front catheter engagement portion 8B is formed into a barrel-shaped (columnar-shaped) configuration. With lengthwise sides of the front catheter engagement portion 8B, is a flat section 13 provided.

It is noted that the carves 21 are not always necessary, and the carves 21 may be omitted depending on the configuration of the front catheter engagement portion 8A (8B). Instead of using the adhesive materials to the front catheter engagement portion 8A (8B), an annular tube may be soldered around the helical spring 6 to form the front catheter engagement portion. Otherwise, the front catheter engagement portion may be formed by depositing multi-layered solder on the helical spring 6.

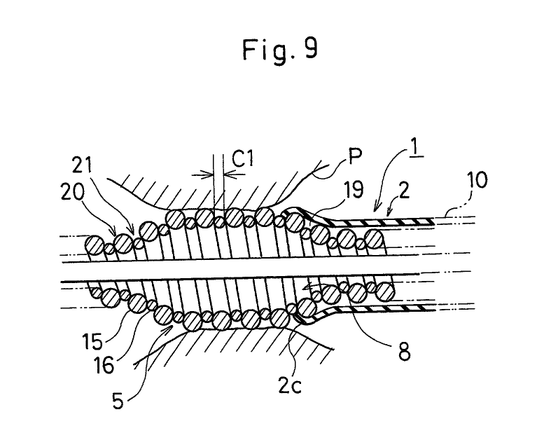

FIG. 9 shows a sixth embodiment of the invention in which the leading bulge portion 5 forms a composite helical spring structure combining a first ellipsoidal helical spring 20 with a second ellipsoidal helical spring 21. Line elements 15 of the first ellipsoidal helical spring 20 is diametrically greater than line elements 16 of the second ellipsoidal helical spring 21.

The former line elements 15 and the latter line elements 16 are alternately arranged tightly with no clearance provided between their neighboring line elements. A rear half section of the leading bulge portion 5 defines the front catheter engagement portion 8.

Upon provisionally connecting the balloon catheter 2 to the front catheter engagement portion 8, an open end section 2c of the balloon catheter 2 is elastically expanded so that the open end section 2c fits into a spiral cavity 19 (i.e., clearance C1) formed between the line elements 15 of the first ellipsoidal helical spring 20.

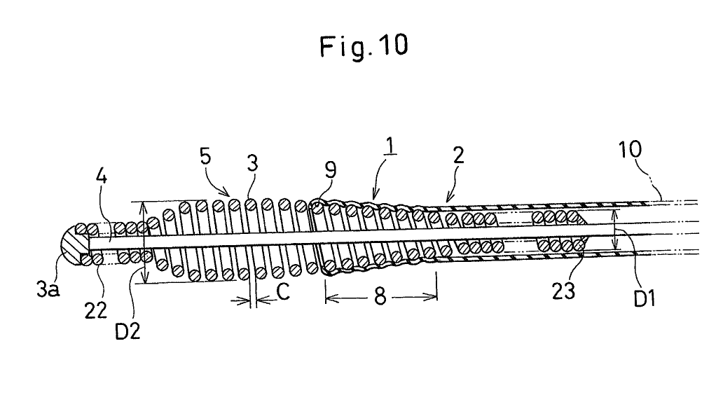

FIG. 10 shows a way how the medical guide wire 1 is introduced into the blood vessel together with the balloon catheter 2. In this instance, a forward helical spring 22 and a rearward helical spring 23 are discretely coiled around the core member 4. The forward helical spring 22, however, can be consecutively extended to be connected to the rearward helical spring 23.

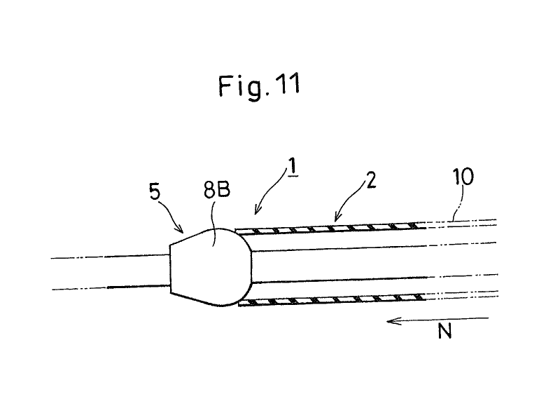

FIG. 11 shows a way how the medical guide wire 1 and the balloon catheter 2 are introduced into the blood vessel separately. The medical guide wire 1 is firstly introduced into the blood vessel, and then the balloon catheter 2 is inserted into the blood vessel in the direction of an arrow N to fit over the front catheter engagement portion 8B which is mirror finished in the same manner as done in the fifth embodiment of the invention (FIG. 7).

In this situation, the front catheter engagement portion 8B readily introduces the balloon portion 10 into the stricture blood vessel area (P) without injuring the blood vessel and the stricture blood vessel area (P).

As understood from the foregoing description, the medical guide wire and the balloon catheter are inserted into the blood vessel with one single step procedure upon placing the balloon portion at the stricture blood vessel area. This enables the manipulator to quickly placing the balloon portion at the stricture blood vessel area with ease and safe, thus alleviating pains the patient suffers at the time of inserting the medical guide wire into the blood vessel to cure the stricture blood vessel area without injuring the blood vessel and the stricture blood vessel area.

It is observed that the carves 12 may be provided with the balloon catheter 2 in the second and third embodiments of the invention (FIGS. 2 and 3).

While there has been described what is at present thought to be preferred embodiments of the invention, it will be understood that modifications may be made therein and it is intended to cover in the appended claims all such modifications which fall within the scope of the invention.

What is claimed is:

1. A medical guide wire comprising:
an elongated and flexible core member;
a front catheter engagement portion provided around said core member and formed into a bulged shape or a truncated cone shape, a diameter of which progressively decreases as approaching a rear end of said front catheter engagement portion;
said front catheter engagement portion being capped with a balloon catheter to provisionally connect said balloon catheter to said front catheter engagement portion so that said balloon catheter is inserted into a blood vessel concurrently at the time when introducing a medical guide wire into said blood vessel.

2. The medical guide wire according to claim 1, wherein a leading bulge portion is formed by an ellipsoidal helical spring or a columnar member, and a rear half of said leading bulge portion forming said front catheter engagement portion so as to introduce said leading bulge portion into a stricture blood vessel area.

3. The medical guide wire according to claim 1, wherein said front catheter engagement portion is formed by depositing a solder or an adhesive on said core member.

4. The medical guide wire according to claim 1, wherein a provisionally connecting member is provided at a front open end of said balloon catheter to provisionally connect said balloon catheter to said front catheter engagement portion.

5. The medical guide wire according to claim 4, wherein said provisionally connecting member is a carve or a rolled end provided at said front open end of said balloon catheter.

6. The medical guide wire according to claim 2, wherein said provisionally connecting member is a groove which fit into said leading bulge portion formed by an ellipsoidal helical spring.

7. The medical guide wire according to claim 1, wherein said balloon catheter and said front catheter engagement portion are formed by a common synthetic resin to produce a coefficient of static friction therebetween, a magnitude of which is determined enough to provisionally connect said balloon catheter to said front catheter engagement portion.

8. The medical guide wire according to claim 7, wherein said balloon catheter and said front catheter engagement portion are commonly formed by a synthetic resin selected from the group consisting of polyamide, polyvinyl chloride, polytetrafluoroethylene and polyethylene.
